SEQUENCE LISTING (50 sequences)

SEQ ID NO: 1 - protein, 101 aa, H. sapiens
MSSAAGFCASRPGLLFLGLLLLPLVVAFASAEAEEDGDLQCLCVKTTSQVRPRHITSLEVIKAGPHCPTAQLIATLKNGRKICLDLQAPLYKKIIKKLLES

SEQ ID NO: 2 - protein, 70 aa, H. sapiens
EAEEDGDLQCLCVKTTSQVRPRHITSLEVIKAGPHCPTAQLIATLKNGRKICLDLQAPLYKKIIKKLLES

SEQ ID NO: 3 - protein, 6 aa, H. sapiens
KKIIKK

SEQ ID NO: 4 - protein, 40 aa, Artificial Sequence
  Protein Fragment
ARKKAAKAARKKAAKAARKKAAKAARKKAAKAVLVLVLVL

SEQ ID NO: 5 - protein, 8 aa, Artificial Sequence
  Protein Fragment
VLVLVLVL

SEQ ID NO: 6 - protein, 8 aa, Artificial Sequence
  Protein Fragment
ILILILIL

SEQ ID NO: 7 - protein, 8 aa, Artificial Sequence
  Protein Fragment
VIVIVIVI

SEQ ID NO: 8 - protein, 12 aa, Artificial Sequence
  Protein Fragment
AAKAVLVLVLVL

SEQ ID NO: 9 - protein, 36 aa, Artificial Sequence
  Protein Fragment
AKKARAAKKARAAKKARAAKKARAAKKARAVLVLVL

SEQ ID NO: 10 - protein, 30 aa, Artificial Sequence
  Protein Fragment
ARKKAAKAARKKAAKAARKKAAKAVLVLVL

SEQ ID NO: 11 - protein, 34 aa, Artificial Sequence
  Protein Fragment
ARKKAAKAARKKAAKAARKKAAKAVLVLVLVLVL

SEQ ID NO: 12 - protein, 24 aa, Artificial Sequence
  Protein Fragment
ARKKAAKAARKKAAKAVLVLVLVL

SEQ ID NO: 13 - protein, 16 aa, Artificial Sequence
  Protein Fragment
ARKKAAKAVLVLVLVL

SEQ ID NO: 14 - protein, 32 aa, Artificial Sequence
  Protein Fragment
ARKKAAKAVLVLVLVLARKKAAKAARKKAAKA

SEQ ID NO: 15 - protein, 32 aa, Artificial Sequence
  Protein Fragment
ARKKAAKAARKKAAKAARKKAAKAILILILIL

SEQ ID NO: 16 - protein, 32 aa, Artificial Sequence
  Protein Fragment
ARKKAAKAARKKAAKAARKKAAKAVIVIVIVI

SEQ ID NO: 17 - protein, 25 aa, Artificial Sequence
  Protein Fragment
VLVLARKKAAKAPARKKAAKAVLVL

SEQ ID NO: 18 - protein, 25 aa, Artificial Sequence
  Protein Fragment
VLVLARKKAAKAPAAAAAAAAVLVL

SEQ ID NO: 19 - protein, 26 aa, Artificial Sequence
  Protein Fragment
VLVLARKKAAKAPARKKAAKADVLVL

SEQ ID NO: 20 - protein, 25 aa, Artificial Sequence
  Protein Fragment
LVLARKKAAKAPAAAAAAAADVLVL

SEQ ID NO: 21 - protein, 21 aa, Artificial Sequence
  Protein Fragment
VLVLAKKARAPAKKARAVLVL

SEQ ID NO: 22 - protein, 21 aa, Artificial Sequence
  Protein Fragment
VLVLAKKARAPAAAAAAVLVL

SEQ ID NO: 23 - protein, 22 aa, Artificial Sequence
  Protein Fragment
VLVLAKKARAPAKKARADVLVL

SEQ ID NO: 24 - protein, 22 aa, Artificial Sequence
  Protein Fragment
VLVLAKKARAPAAAAAADVLVL

SEQ ID NO: 25 - protein, 14 aa, Artificial Sequence
  Protein Fragment
VLVLARKKAAKACA

SEQ ID NO: 26 - protein, 16 aa, Artificial Sequence
  Protein Fragment
VLVLARKKAAKACADA

SEQ ID NO: 27 - protein, 12 aa, Artificial Sequence
  Protein Fragment
VLVLAKKARACA

SEQ ID NO: 28 - protein, 14 aa, Artificial Sequence
  Protein Fragment
VLVLAKKARACADA

SEQ ID NO: 29 - protein, 4 aa, Artificial Sequence
  Protein Fragment
CADA

SEQ ID NO: 30 - protein, 4 aa, Artificial Sequence
  Protein Fragment
VLVL

SEQ ID NO: 31 - protein, 6 aa, Artificial Sequence
  Protein Fragment
VLVLVL

SEQ ID NO: 32 - protein, 8 aa, Artificial Sequence
  Protein Fragment
ARKKAAKA

SEQ ID NO: 33 - protein, 32 aa, Artificial Sequence
  Protein Fragment
ARKKAAKAAAAAAAAAAAAAAAAAARKKAAKA

SEQ ID NO: 34 - protein, 32 aa, Artificial Sequence
  Protein Fragment
ARKKAAKAARKKAAKAARKKAAKAARKKAAKA

SEQ ID NO: 35 - protein, 36 aa, Artificial Sequence
  Protein Fragment
AKKARAAKKARAAKKARAAKKARAAKKARAAKKARA

SEQ ID NO: 36 - protein, 32 aa, Artificial Sequence
  Protein Fragment
ARKKAAKAARKKAAKAARKKAAKAARKKCAKA

SEQ ID NO: 37 - protein, 34 aa, Artificial Sequence
  Protein Fragment
CARKKAAKAARKKAAKAARKKAAKAARKKAAKAC

SEQ ID NO: 38 - protein, 4 aa, Artificial Sequence
  Protein Fragment
ILIL

SEQ ID NO: 39 - protein, 6 aa, Artificial Sequence
  Protein Fragment
ILILIL

SEQ ID NO: 40 - protein, 36 aa, Artificial Sequence
  Protein Fragment
ARKKAAKAARKKAAKAARKKAAKAVLVLVLVLVLVL

SEQ ID NO: 41 - protein, 36 aa, Artificial Sequence
  Protein Fragment
VLVLARKKAAKAARKKAAKAARKKAAKAARKKCAKA

SEQ ID NO: 42 - protein, 4 aa, Artificial Sequence
  Protein Fragment
VIVI

SEQ ID NO: 43 - protein, 6 aa, Artificial Sequence
  Protein Fragment
VIVIVI

SEQ ID NO: 44 - protein, 6 aa, Artificial Sequence
  Protein Fragment
AKKARA

SEQ ID NO: 45 - protein, 8 aa, Artificial Sequence
  Protein Fragment
LLLLLLLL

SEQ ID NO: 46 - protein, 8 aa, Artificial Sequence
  Protein Fragment
VFVFVFVF

SEQ ID NO: 47 - protein, 12 aa, Artificial Sequence
  Protein Fragment
VLVLVLVLVLVL

SEQ ID NO: 48 - protein, 32 aa, Artificial Sequence
  Protein Fragment
ARKKAAKAARKKAAKAARKKAAKALLLLLLLL

SEQ ID NO: 49 - protein, 32 aa, Artificial Sequence
  Protein Fragment
ARKKAAKAARKKAAKAARKKAAKAVFVFVFVF

SEQ ID NO: 50 - protein, 40 aa, Artificial Sequence
  Protein Fragment
ARKKAAKAARKKAAKAVLVLVLVLARKKAAKAARKKAAKA

(19) United States
(12) Patent Application Publication
(10) Pub. No.: US 2014/0038205 A1
(43) Pub. Date: Feb. 6, 2014

(54) METHODS AND COMPOSITIONS RELATING TO COAGULATION ASSAYS

(71) Applicant: Steven Jay Raynard, Halifax (CA)

(72) Inventor: Steven Jay Raynard, Halifax (CA)

(73) Assignee: Precision BioLogic Inc., Dartmouth (CA)

(21) Appl. No.: 13/984,335

(22) PCT Filed: Jan. 18, 2012
(86) PCT No.: PCT/CA12/50031

§ 371 Date: Oct. 9, 2013

Related U.S. Application Data
(60) Provisional application No. 61/444,550, filed on Feb. 18, 2011.

(51) Int. Cl. G01N 33/86

(52) U.S. Cl. CPC G01N 33/86
USPC 435/7.8

(57) ABSTRACT

The invention provides methods and compositions relating to the detection and neutralization of heparin and heparin derivatives in vivo and in vitro. To neutralize heparin in a patient sample, a composition comprising a complex of a heparin-binding agent and a carrier compound is used prior to performing coagulation assays.

### RELATED APPLICATIONS

This application claims the benefit under 35 U.S.C. §119(e) of U.S. Provisional Application Ser. No. 61/444,550, entitled “METHODS AND COMPOSITIONS RELATING TO COAGULATION ASSAYS” filed on Feb. 18, 2011, which is incorporated by reference herein in its entirety.

### BACKGROUND OF INVENTION

Heparin is widely used in hospital environments as a broad spectrum anticoagulant for patient management as well as a flush to prevent indwelling catheters from clotting out. As a result, one of the most common sources of pre-analytical error in the clinical coagulation laboratory is contamination of the patient sample with heparin. Typically, heparin contamination is manifested in vitro by a prolonged activated partial thromboplastin time (aPTT). This is problematic for labs that receive their samples from off-site collection because they are unable to call the phlebotomist to verify how the sample was collected. Despite best practices that require samples for coagulation testing to be collected in a very specific manner, many nurses and phlebotomists will collect the sample through an indwelling catheter from the patient. These catheters are kept open by flushing them with heparin, so this creates a high likelihood for heparin contamination in the sample.

### SUMMARY OF INVENTION

The invention is premised in part on the discovery of a complex comprising a heparin-binding agent with unexpected properties. The complex can be used to neutralize heparin in vitro or in vivo without contributing any anti- or pro-coagulant effect itself. As described herein, heparin neutralization is an important process in vitro and in vivo. Heparin presence in a patient sample can lead to an incorrect result from an in vitro analysis such as a coagulation analysis. Heparin in vivo, while necessary in some instances, should be controlled to prevent unwanted anti-coagulant activity. The compositions and methods of the invention are useful in both regards. Significantly, the complex is able to bind to heparin when it is present. However, when heparin is absent, the complex remains unperturbed and does not interact with other molecules including factors involved in the coagulation pathway. As a result, the use of the complex itself does not lead to spurious results in the absence of heparin.

The invention therefore provides in one aspect a method comprising adding, to a patient sample in vitro, a composition comprising a complex of a heparin-binding agent and a carrier compound, wherein the heparin-binding agent has a higher binding affinity for heparin than for the carrier compound, and measuring coagulation activity in the patient sample. In some embodiments, coagulation activity in the presence of the complex is compared to coagulation activity in the absence of the complex. In some embodiments, coagulation activity that is greater in the presence of the complex than in the absence of the complex indicates presence of heparin or a heparin derivative in the patient sample.

In another aspect, the invention provides a method comprising performing a coagulation assay, on a patient sample, in the presence of a heparin-binding agent complexed to a carrier compound, wherein the heparin-binding agent complexed to the carrier compound is allogeneic to the patient sample and wherein the heparin-binding agent has a higher binding affinity for heparin than for the carrier compound. In some embodiments, the method further comprises performing another coagulation assay, using another aliquot of the patient sample, in the absence of the heparin-binding agent complexed to the carrier compound. In some embodiments, the method further comprises comparing results from the coagulation assay in the presence of the heparin-binding agent complexed to the carrier compound to results from a coagulation assay performed in the absence of the heparin-binding agent complexed to the carrier compound.

In another aspect, the invention provides a method for detecting heparin or a heparin derivative in a patient sample comprising measuring coagulation activity in a patient sample in the absence and presence of an exogenous complex of a heparin-binding agent and a carrier compound, wherein the heparin-binding agent has a higher binding affinity for heparin than for the carrier compound, wherein a higher coagulation activity in the presence of the exogenous complex than in the absence of the exogenous complex indicates presence of heparin or a heparin derivative in the patient sample.

Various aspects apply equally to various aspects described herein. For example, in some embodiments, wherein the patient sample is suspected of containing heparin or a heparin derivative. In some embodiments, the patient sample is characterized as having a prolonged clotting time, in the absence of the heparin-binding agent complexed to the carrier compound. In some embodiments, the patient sample is not known to contain heparin or a heparin derivative. In some embodiments, the patient sample is known to contain or is suspected of containing heparin or a heparin derivative.

In some embodiments, the coagulation assay is an activated partial thromboplastin time (aPTT) assay. In some embodiments, the coagulation assay is an FII, FV, FVIII, FIX, FX, FXI or FXII activity assay. In some embodiments, the coagulation assay is an activated protein C (APC) resistance assay. In some embodiments, the coagulation assay is a Bethesda assay. In some embodiments, the coagulation assay is a prothrombin time (PT) assay. In some embodiments, the coagulation assay is dilute Russell's viper venom time (dRVVT) assay. In some embodiments, the coagulation assay is a hexagonal (II) phase phospholipid-based assay. In some embodiments, the coagulation assay is an activated clotting time (ACT) assay. In some embodiments, the coagulation assay is a thromboelastograph (TEG) tracing. In some embodiments, the coagulation assay is a thrombin generation assay. In some embodiments, the coagulation assay is a protein C assay. In some embodiments, the coagulation assay is a protein S assay.

In some embodiments, the patient sample is a blood sample. In some embodiments, the patient sample is a plasma sample.

In another aspect, the invention provides a method comprising administering to a subject receiving heparin therapy an isolated complex of a heparin-binding agent and a carrier compound, wherein the heparin-binding agent has a higher binding affinity for heparin than for the carrier compound, in an effective amount to reduce heparin level in the subject.

In some embodiments, heparin is unfractionated heparin. In some embodiments, heparin is low molecular weight heparin. In some embodiments, the heparin derivative is a heparinoid.

In another aspect, the invention provides a method comprising contacting a patient sample to a complex of a heparin-binding agent and a carrier compound, wherein the heparin-binding agent is bound to a solid support, and wherein the heparin-binding agent has a higher binding affinity for heparin than for the carrier compound. In some embodiments, the method further comprises detecting binding of heparin or a heparin derivative to the heparin-binding agent. In some embodiments, the method further comprises detecting dissociation of the carrier compound from the heparin-binding agent.

In another aspect, the invention provides a composition comprising a complex of a heparin-binding agent and a carrier compound, immobilized on a solid support, wherein the heparin-binding agent has a lower binding affinity for the carrier compound than heparin, wherein the solid support is not an affinity chromatography column.

In another aspect, the invention provides a composition comprising a complex of a heparin-binding agent and a carrier compound, immobilized on a solid support, wherein the heparin-binding agent has a lower binding affinity for the carrier compound than heparin, and wherein the heparin-binding agent and/or the carrier compound are bound to a detectable label.

In another aspect, the invention provides a composition comprising a synthetic complex of a heparin-binding agent and a carrier compound wherein the heparin-binding agent has a lower binding affinity for the carrier compound than heparin, and a preservative.

In another aspect, the invention provides a composition comprising a synthetic complex of a heparin-binding agent and a carrier compound wherein the heparin-binding agent has a lower binding affinity for the carrier compound than heparin, wherein the composition is present in an evacuated vacuum venipuncture collection tube.

In another aspect, the invention provides a composition comprising a synthetic complex of a heparin-binding agent and a carrier compound wherein the heparin-binding agent has a lower binding affinity for the carrier compound than heparin, wherein the composition is not sterile.

In another aspect, the invention provides a composition comprising a synthetic complex of a heparin-binding agent and a carrier compound wherein the heparin-binding agent has a lower binding affinity for the carrier compound than heparin, wherein the composition is not pharmaceutically acceptable.

In another aspect, the invention provides a composition comprising a heparin-binding agent, a carrier compound wherein the heparin-binding agent has a lower binding affinity for the carrier compound than heparin, and a patient sample allogeneic to the heparin-binding agent.

Various aspects apply equally to various aspects described herein, and these are described in greater detail below.

In some embodiments, the heparin-binding agent is a heparin-binding protein. In some embodiments, the heparin-binding agent is a heparin-binding polymer.

In some embodiments, the heparin-binding agent is naturally occurring. In some embodiments, the heparin-binding agent is isolated from a naturally occurring source. In some embodiments, the heparin-binding agent is isolated from platelets. In some embodiments, the heparin-binding agent is isolated from human platelets.

In some embodiments, the heparin-binding agent is recombinantly produced. In some embodiments, the heparin-binding agent is recombinantly produced in mammalian cells.

In some embodiments, the heparin-binding agent is platelet factor 4 (PF4). In some embodiments, the heparin-binding agent is a heparin-binding platelet factor 4 (PF4) fragment. In some embodiments, the heparin-binding agent is poly-L-lysine. In some embodiments, the heparin-binding agent is protamine sulfate. In some embodiments, the heparin-binding agent is hexadimethrine bromide (Polybrene®).

In some embodiments, the carrier compound is a proteoglycan. In some embodiments, the carrier compound is a glycosaminoglycan. In some embodiments, the carrier compound is polyanionic. In some embodiments, the carrier compound is chondroitin sulfate. In some embodiments, the carrier compound is chondroitin-4-sulfate. In some embodiments, the carrier compound is dermatan sulfate. In some embodiments, the carrier compound is keratan sulfate.

In some embodiments, the carrier compound is heparan sulfate.

In some embodiments, the heparin-binding agent is platelet factor 4 (PF4) and the carrier compound is chondroitin sulfate. In some embodiments, the heparin-binding agent is platelet factor 4 (PF4) and the carrier compound is chondroitin-4-sulfate.

In some embodiments, the carrier compound is bound to a detectable label. In some embodiments, the heparin-binding agent is bound to a detectable label. In some embodiments, the detectable label is a fluorophore.

In some embodiments, the heparin-binding agent is bound to a FRET donor and the carrier compound is bound to a FRET acceptor. In some embodiments, the heparin-binding agent is bound to a FRET acceptor and the carrier compound is bound to a FRET donor. In some embodiments, the heparin-binding agent is bound to a fluorophore and the carrier compound is bound to a quencher.

In some embodiments, the heparin-binding agent is in solution. In some embodiments, the heparin-binding agent is immobilized on a solid support. In some embodiments, the carrier compound is immobilized on a solid support. In some embodiments, the solid support is an inside surface of a container. In some embodiments, the solid support is a thin film. In some embodiments, the solid support is a filter. In some embodiments, the solid support is a dipstick. In some embodiments, the solid support is a microparticle.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be to appreciated that terminology explicitly employed herein that also may appear in any disclosure incorporated by reference should be accorded a meaning most consistent with the particular concepts disclosed herein.

### BRIEF DESCRIPTION OF DRAWINGS

FIG. 1 is a graph showing the ability of purified PF4 to neutralize unfractionated heparin in the aPTT assay.

FIG. 2 is a graph showing the effect of PF4 on the aPTT of non-heparinized plasma.

FIG. 3 is a bar graph showing the effect of a complex of PF4 chondroitin-4-sulfate (C4S) on the aPTT of non-heparinized plasma (NP).

FIG. 4 is a bar graph showing neutralization of unfractionated heparin (UFH) anticoagulant activity by the complex of PF4 and chondroitin-4-sulfate (C4S).

### DETAILED DESCRIPTION OF INVENTION

Heparin is used extensively in medicine in order to prevent or reduce coagulation (clotting) activity in a subject. Heparin is used to flush indwelling catheters, including central lines from which blood samples are drawn, in order to prevent clotting of blood therein. Heparin therapy is used therapeutically to prevent or reduce the likelihood of blood clot formation in subjects. Such blood clots can lead to pulmonary embolisms, as an example.

The ability to detect the presence of heparin in a patient sample, and optionally to eliminate heparin or to control its level is useful in vitro and in vivo. For example, the presence of heparin in a patient sample such as a blood or plasma sample can lead to incorrect results in a coagulation assay (i.e., the presence of heparin can result in reduced coagulation activity readings, such as increased coagulation times). Accordingly, the ability to determine whether heparin is present in the sample, and in some instances to also neutralize heparin in the sample at the same time, can result in an accurate analytical readout. The ability to do this in real time using a single sample from a patient, rather than requiring another sample, is also advantageous. When used in vivo, it is important to control the level of heparin since levels that are too high or too low can have unintended consequences, including uncontrolled bleeding and abnormal clot formation respectively.

Commonly used methods used for removing or neutralizing heparin in vivo or in vitro have included the use of agents such as protamine sulfate and hexadimethrine bromide (Polybrene®), the heparin-degrading enzyme heparinase (Hepzyme™), and extracorporeal heparin removal devices. However, each of these techniques has been associated with its own set of drawbacks (Cumming et al. 1986).

The invention provides compositions that can be used to bind heparin and thereby neutralize its activity. The invention also provides methods of use for these compositions including in vitro and in vivo methods of use.

Complex of Heparin-Binding Agent and Carrier Compound

The compositions of the invention comprise complexes of a heparin-binding agent and a carrier compound. The heparin-binding agent is reversibly complexed to the carrier compound.

Accordingly, such complexes are non-covalent in nature. As described in greater detail below, the heparin-binding agent has a higher binding affinity for heparin than it does for a carrier compound of the invention. Therefore, in the presence of heparin, the heparin-binding agent dissociates from the carrier compound and binds to heparin. In the absence of heparin, the heparin-binding agent remains complexed to the carrier compound. It has been found according to the invention that such a complex is advantageous particularly if the heparin-binding agent, in an uncomplexed state, can influence coagulation readouts.

The complex may be prepared by simply combining the heparin-binding agent and the carrier compound. The molar amounts of heparin-binding agent and carrier compound present in a complex will vary depending on the nature of the heparin-binding agent and the carrier compound. In some instances, the molar ratio of heparin-binding agent to carrier compound will be 1:1, 1:2, 1:3, 1:4, 1:5, 1:6, 1:7, 1:8, 1:9, 1:10, or more. In other instances, the molar ratio of heparin-binding agent to carrier compound will be 2:1, 3:1, 4:1, 5:1, 6:1, 7:1, or 8:1 or greater. In some embodiments, the molar ratio of heparin-binding agent to carrier compound will range from about 5:1 to about 8:1.

Complexes of the invention include without limitation PF4 comprising complexes such as PF4 and chondroitin sulfate complexes, PF4 and chondroitin-4-sulfate complexes, PF4 and chondroitin-6-sulfate complexes, PF4 and dermatan sulfate complexes, PF4 and keratan sulfate complexes, and PF4 and heparan sulfate complexes; poly-lysine comprising complexes such as poly-lysine and chondroitin sulfate complexes, poly-lysine and chondroitin-4-sulfate complexes, to poly-lysine and chondroitin-6-sulfate complexes, poly-lysine and dermatan sulfate complexes, poly-lysine and keratan sulfate complexes, and poly-lysine and heparan sulfate complexes; protamine comprising complexes such as protamine and chondroitin sulfate complexes, protamine and chondroitin-4-sulfate complexes, protamine and chondroitin-6-sulfate complexes, protamine and dermatan sulfate complexes, protamine and keratan sulfate complexes, and protamine and heparan sulfate complexes; and Polybrene® (hexadimethrine bromide) comprising complexes such as Polybrene® and chondroitin sulfate complexes, Polybrene® and chondroitin-4-sulfate complexes, Polybrene® and chondroitin-6-sulfate complexes, Polybrene® and dermatan sulfate complexes, Polybrene® and keratan sulfate complexes, and Polybrene® and heparan sulfate complexes.

The complexes may be formed using heparin-binding agents and carrier compounds that are naturally occurring or non-naturally occurring, whether obtained from naturally occurring sources (e.g., isolated from naturally occurring sources) or prepared synthetically. As described in greater detail below, the source of the complex or either of its components typically will not be the patient from whom a sample is obtained for coagulation activity testing.

The complex may be prepared in any solution that does not negatively impact the function and structure of the components. Examples include water, saline solution, and a buffered saline solution. When used in vivo, the composition comprising the complex should be sterile and non-toxic. When used in vitro, the composition comprising the complex may be non-sterile and/or it may contain additional components that are not suitable for in vivo use (e.g., it may comprise non-pharmaceutically acceptable components). For example, the composition may comprise a preservative, a buffer not suitable for in vivo use, salts not suitable for in vivo use, one or more reagents used in coagulation assays such as those described herein, and the like. Coagulation assay reagents may depend upon the coagulation assay used. Examples include without limitation phospholipids, divalent cations (e.g., calcium), surface activator (e.g., kaolin, micronized silica, celite, ellagic acid), tissue factor, protein C activator (e.g., Protac®, thrombin, thrombomodulin), dilute Russell's viper venom, factor deficient plasma, platelet poor plasma, ecarin, and platelet activator (e.g., arachidonic acid, collagen, epinephrine, ADP).

In some embodiments, the complex or a composition comprising the complex may be provided in a multiple use container in contrast to a single use container such as a single use vial typically used for in vivo uses. In some embodiments, the complex or composition comprising the complex may be provided in a container that is not sterile and/or not suitable for in vivo use. In still other embodiments, it may be provided in an evacuated vacuum venipuncture collection tube, such as Vacutainer®. It may be included, for example, in a solution or in a semi-solid (e.g., a matrix) contained in the tube. Additionally or alternatively, it may be provided as a coating on the interior surface of the tube or on the interior surface of the lid or closure of the tube.

The invention contemplates “batch” compositions that comprise the complexes, and other constituents at concentrations that would be toxic if used in vivo, and that are diluted when used in vitro. The composition comprising the complex may, in some embodiments, be non-isotonic with the patient sample being tested, including a plasma sample.

The complex may be provided as a solid including a powder such as a lyophilized powder or in solution including a cryopreserved solution, according to some embodiments. In other embodiments, the complex is attached to a solid support such as a dipstick, a filter such as a nitrocellulose filter, or microparticles (e.g., glass, silica or polystyrene microspheres). In some embodiments, the complex is not provided on an affinity column.

It has been reported that complexes of PF4 and chondroitin-4-sulfate may be naturally present in human blood samples. Accordingly, in various embodiments, the complexes of the invention may be referred to as exogenous complexes. This intends that the complex is one added to (or combined with) a patient sample and it is not inherent to the patient sample (i.e., it is not a naturally occurring complex in the patient sample). Similarly, the complexes may be referred to as allogeneic to the patient sample, again intending that the complex derives from a source other than the patient and thus is not present in the patient sample prior to addition or combination. Reference to the complexes of the invention being isolated, extrinsic to the patient sample, or synthetic also indicate that the complex is not the naturally occurring complex that may be present in the patient sample. It should also be clear that the invention contemplates complexes that could not be naturally occurring because one or both components of the complex are not naturally occurring.

Heparin-Binding Agent

The heparin-binding agent is an agent that binds to heparin and, with less affinity, to a carrier compound of the invention. The heparin-binding agent may have a binding affinity for heparin that is 10%, 20%, 30%, 40%, or 50% higher than its binding affinity for the carrier compound. In some instances, it may have a binding affinity for heparin that is 2-fold, 3-fold, 4-fold, 5-fold, 10-fold, 20-fold, 50-fold, or 100-fold greater than its binding affinity for the carrier compound.

The binding activity of a heparin-binding agent may be known a priori and/or it may be measured using standard assays known in the art. Similarly, the binding affinity of a heparin-binding agent for heparin compared to a carrier compound of the invention may be known a priori and/or it may be measured using standard assays known in the art including but not limited to competition binding assays.

The heparin-binding agent may be positively charged at neutral or physiological pH.

The heparin-binding agent may be a polymer such as a protein. Accordingly, it may be referred to as a heparin-binding protein or a heparin-binding polymer. A polymer is an agent comprised of monomers attached to one another, typically but not necessarily in a linear manner. Proteins and peptides are polymers comprised of amino acid monomers. Proteins may be comprised of single peptide chains or multiple peptide chains and such multiple chains may be complexed with each other covalently or non-covalently, directly or indirectly. The heparin-binding polymer may comprise (i.e., include) or consist of monomers of the same or different classes (e.g., all the monomers may be amino acids).

The heparin-binding agent may be naturally occurring. A naturally occurring agent is one that can be found in nature from a naturally occurring source. It may be isolated from the naturally occurring source, but it is not so limited. As an example, a naturally occurring agent may be synthesized in vitro yet may be identical to the naturally occurring form of the agent.

An isolated heparin-binding agent is an agent that is physically separated from its normal environment. If the heparin-binding agent is obtained from an in vivo source such as but not limited to a naturally occurring cell or organism, then it is isolated when it is physically separated from the other components present in that in vivo source. It may be partially or completely separated from one or more components. If the agent is sufficiently isolated, it may be referred to herein as purified. The level of purification may also be stated. For example, the level of purification may be expressed as the amount of agent in a composition based on a weight by weight, volume by volume, weight by volume, or specific activity measurement. In some embodiments, the heparin-binding agent is isolated from platelets, including human to platelets.

The heparin-binding agent may be non-naturally occurring. A non-naturally occurring agent is one that is not found in nature from a naturally occurring source. Such an agent will be synthesized in vitro or in vivo using manipulated sources (e.g., genetically manipulated sources). Such non-naturally occurring agents may be similar but not identical to naturally occurring agents. For example, they may differ from naturally occurring versions of the agent with respect to glycosylation levels and patterns, phosphorylation levels and patterns, the presence of non-naturally occurring monomers such as amino acids in the case of proteins or peptides, and the like. Agents made in bacteria, as an example, typically have different glycosylation characteristics than agents made in eukaryotic cells such as mammalian cells.

The heparin-binding agent may be recombinantly produced in bacterial or eukaryotic cells such as mammalian cells. Recombinant production of proteins or peptides will be discussed in greater detail below. Briefly, a protein or peptide is typically recombinantly produced by introducing into a cell nucleic acids that code for the protein or peptide along with nucleic acids that control the transcription of those nucleic acids and the translation of resultant mRNA into the desired protein or peptide. Agents may also be recombinantly produced using cell free systems, as is known in the art. In certain embodiments, the heparin-binding agents are recombinantly produced in mammalian cells.

In some embodiments, the heparin-binding agent is provided at a concentration that, if used in the absence of the carrier compound, would have anti- or pro-coagulant activity itself. Accordingly, use of the heparin-binding agent complexed to the carrier compound avoids this undesired effect.

Examples of heparin-binding agents include without limitation platelet factor 4 (PF4), heparin-binding PF4 fragments, PF4 variants such as PF4 fusion proteins, poly-lysine (i.e., poly-L-lysine), protamine (e.g., protamine sulfate), heparin-binding protamine fragments, hexadimethrine bromide (i.e., Polybrene®).

PF4 is a protein normally produced and secreted by platelets. In its mature state, it is a 70 amino acid protein having a molecular weight of about 7.8 kDa. It has been isolated and cloned. (Deuel et al. PNAS 74(6):2256-2258, 1977.) Human PF4 mRNA sequence can be found at GenBank Accession No. NM_{—}002619. Human PF4 precursor protein sequence can be found at GenBank Accession No. NP_{—}002610. The precursor sequence is as follows: MSSAAGFCAS RPGLLFLGLL LLPLVVAFAS AEAEEDGDLQ CLCVKTTSQV RPRHITSLEV IKAGPHCPTA QLIATLKNGR KICLDLQAPL YKKIIKKLLE S (SEQ ID NO: 1), where the underlined sequence represents the amino acid sequence of the mature protein (SEQ ID NO:2), the sequence that precedes the mature sequence is the signal peptide sequence that is present in the precursor, and the bolded sequence is the minimal heparin-binding domain of PF4 (SEQ ID NO:3).

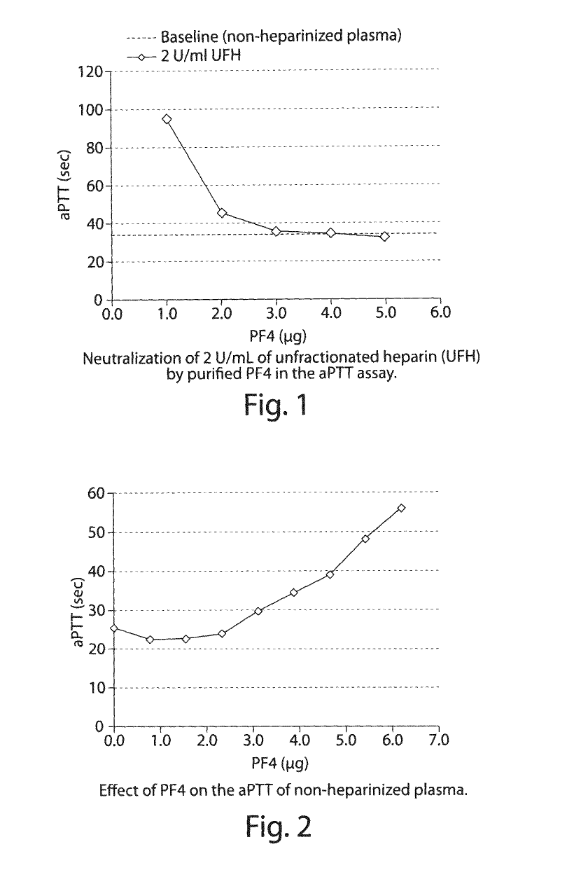

PF4 tetramers form a cylindrical structure displaying an equatorial ring of positively charged amino acids around which the polyanionic heparin molecules wrap and bind with exceptionally high affinity. The formation of a PF4-heparin complex strongly impairs the capacity of heparin to bind AT and stimulate its anticoagulant activity. FIG. 1 shows the neutralization of 2 U/mL of unfractionated heparin (UFH) by PF4 purified from human source platelets as assessed in the aPTT assay.

PF4 can be isolated from naturally occurring sources such as platelets and serum. An exemplary method for isolating PF4 from platelets is provided by Rucinski et al. Blood 53:47-62, 1979 and in U.S. Pat. No. 4,702,908 to Thorbecke et al. These isolation methods are incorporated by reference herein. Briefly, these methods involve (1) washing, preferably fresh, platelets with a standard buffer (e.g., a pyrogen-free, sterile buffer) and resuspending the washed platelets in the same buffer at a density of 2×10⁹ platelets/ml, (2) adding thrombin (e.g., 1 U/ml) or A23187 (e.g., 250 nM), or arachidonic acid (e.g., 50 μM) to release PF4 from the platelets, (3) incubating the suspension for 1 hour and then centrifuging the suspension, (4) applying the supernatant to a heparin-agarose column equilibrated with NaCl/Tris (e.g., 0.5M NaCl, 0.05M Tris, pH 8.0) and washing the column until protein is no longer eluted, (5) eluting PF4 using 1.5M NaCl, 0.05M Tris pH 8.0 solution.

PF4 can be produced recombinantly by introducing its coding sequence into a bacterial or mammalian expression system, using methods known in the art. For example, the PF4 coding nucleic acid, in one embodiment, may be operably linked to a gene expression sequence which directs the expression of the PF4 coding nucleic acid within a cell such as a eukaryotic cell. The “gene expression sequence” is any regulatory nucleotide sequence, such as a promoter sequence or promoter-enhancer combination which facilitates the efficient transcription and translation of the PF4 encoding nucleic acid to which it is operably linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, beta-actin promoter and other constitutive promoters. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art. In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined PF4 encoding nucleic acid. The gene expression sequences optionally include enhancer sequences or upstream activator sequences as desired.

The invention further contemplates heparin-binding PF4 fragments and PF4 variants such as heparin-binding PF4 fusion proteins as heparin-binding agents.

Heparin-binding PF4 fragments are fragments of full-length PF4 that minimally include the heparin-binding domain of PF4. The heparin-binding domain of PF4 minimally comprises residues 61-66 of PF4 having the amino acid sequence of KKIIKK (SEQ ID NO: 3). The heparin-binding PF4 fragments may comprise additional PF4 amino acid sequence at the amino and/or carboxy terminals of the heparin-binding domain, including 1, 2, 3, 4, 5, 6, 7, 8, 9, 10, or more (up to 60) amino acid residues at the amino end, and/or 1, 2, 3 or 4 amino acid residues at the carboxy end. The PF4 fragments may be 1-6, 1-7, 1-8, 1-9, 1-10, 1-20, 1-30, 1-40, 1-50, 1-60, 61, 62, 63, 64, 65, 66, 67, 68, or 69 amino acids in length.

It is to be understood that the invention also contemplates that these heparin-binding PF4 fragments may be modified by the addition of, for example, non-PF4 additional amino acid residues at either, both, or between the ends of the fragment. These fragments may be referred to as heparin-binding PF4 variants. Heparin-binding PF4 variants share some but not total identity with full length naturally occurring PF4 or heparin-binding PF4 fragments. For example, such variants may be about 80%, about 85%, about 90%, about 91%, about 92%, about 93%, about 94%, about 95%, about 96%, about 97%, about 98%, or about 99% identical to full length PF4 or to a heparin-binding PF4 fragment. Variants may comprise a PF4 fragment and additional flanking constituents at the amino and/or carboxy end of the fragment. Such constituents may be amino acid in nature but they are not so limited. In all instances, the variants bind to heparin and the carrier compound and have a greater affinity for heparin than the carrier compound.

Heparin-binding agents may be PF4 fusion proteins. A fusion protein, as used herein, is a protein that contains peptide regions from at least two different proteins. For example, a PF4 fusion protein contains amino acid sequence from PF4 and at least one non-PF4 protein. Such fusion proteins can be formed by fusing, usually at the nucleotide level, coding sequence from PF4 to coding sequence from a non-PF4 protein. Examples of PF4 fusion proteins include PF4 GST fusion protein, PF4 Fc fusion protein, PF4 beta-galactosidase fusion protein, PF4 poly-His fusion protein, and PF4 GFP fusion protein. Fc fusion proteins may comprise regions of the Ig constant domain, including without limitation the hinge region, the CH1 domain, the CH2 domain, and/or CH3 domain, optionally conjugated to the PF4 fragment via the hinge domain. The Fc portion may derive from human antibodies or non-human antibodies. The antibodies may be IgG1 or IgG2, although they are not so limited. Methods of making Fc fusion proteins are known in the art and are described at least in EP0464533.

The heparin-binding agent may be a functionally equivalent peptide analog of PF4. As used herein and in the context of the invention, the term functionally equivalent peptide analog refers to a peptide analog that is capable of binding heparin and preferably that binds to heparin with an affinity that is greater than its affinity for a carrier compound of the invention. Functionally equivalent peptide analogs of PF4 may be identified, for example, using in vitro adhesion assays that measure the ability of the peptide analog to full-length PF4 binding to heparin. Exemplary functionally equivalent peptide analogs of PF4 include analogs of full length PF4 or a PF4 fragment that comprises conservative amino acid substitutions relative to the wild-type sequence provided herein.

Poly-lysine (i.e., poly-L-lysine) may be used as a heparin-binding agent. Poly-lysine is a homopolymer of lysine residues. It may be used in a naturally occurring form or it may be a non-naturally occurring form. Naturally occurring forms, produced through the fermentation by bacteria, are about 25-30 lysine residues in length. Non-naturally occurring forms may be shorter than 25 lysine residues or they may be longer than 30 residues. It is commercially available from Sigma Aldrich and other vendors.

Protamine is a cationic, typically arginine-rich peptide or protein that may be naturally occurring or non-naturally occurring. Naturally occurring protamines include human PRM1 and PRM2, and non-human forms such as salmine, clupeine, iridine, thinnine, stelline, and scylliorhinine. Protamine may be isolated from natural sources such as certain fish species or it may be produced recombinantly. It is typically formulated as a salt such as protamine sulfate (CAS No. 53597-25-4). It is commercially available from Sigma Aldrich and other vendors. The invention further contemplates the use of heparin-binding protamine fragments. Methods for preparing protamine fragments and examples of protamine fragments are provided in published PCT application WO 2006/055196.

Polybrene® is a cationic polymer, also referred to as hexadimethrine bromide (CAS No, 28728-55-4). It is commercially available from Sigma Aldrich, Millipore, Santa Cruz Biotechnology, Inc. and other vendors.

Heparin-binding peptides such as those described in published application US 20060172931 to San Antonio et al. may be used as heparin-binding agents. These peptides are defined by the formulae
R₁(X₁B₁B₂X₂B₃X₃Y₁R₂)ₙR₃, or
R₁(X₁B₁B₂B₃X₂X₃B₄X₄Y₁R₂)ₙR₃,

wherein X₁, X₂, X₃ and X₄ are independently selected from the group consisting of hydropathic amino acids; B₁, B₂, B₃ and B₄ are independently selected from the group consisting of basic amino acids; Y₁ is independently (i) zero amino acids residues or (ii) one to ten amino acid residues, wherein at least one of said amino acid residues is proline; n is an integer from one to ten; R₁, R₂ and R₃ are independently selected segments containing from zero to twenty amino acid residues, provided that at least one of the segments R₁, R₂ or R₃ comprises at least one hydrophobic amino acid residue. Examples include without limitation the various peptide sequences disclosed and referred to as sequences 1-47 in US 20060172931 (corresponding to SEQ ID NOs: 4-50, herein), all of which are incorporated by reference herein. Some embodiments of the invention exclude the heparin-binding peptides of US 20060172931 as heparin-binding agents.

Carrier Compound

The complexes of the invention further comprise a carrier compound. The carrier compound of the invention is an agent that binds to a heparin-binding agent of the invention with lower affinity than does heparin. Accordingly, in the absence of heparin, the carrier compound is bound to the heparin-binding agent. In the presence of heparin, the carrier is competed away from the heparin-binding agent by heparin.

The carrier compounds include agents that are negatively charged at neutral or physiological pH. They may be polyanionic compounds. Carrier compounds of the invention may be glycosaminoglycans (GAG).

As used herein, the carrier compound is not naturally occurring heparin such as unfractionated heparin, low molecular weight heparin, and heparin derivatives, since by definition the heparin-binding agent binds differentially to heparin and the carrier compound.

In some instances, the carrier compound is not an active agent (i.e., when used alone at any dose, or when used alone at the dose that would be released in vivo in the presence of heparin, the carrier compound would have no discernable effect on the subject), nor is it a diagnostic agent (i.e., when used alone at any dose or when used alone at the dose that would be released in vivo in the presence of heparin, the carrier compound would not function as a diagnostic agent). Accordingly, the carrier compound when used alone is not a therapeutic agent and not a prophylactic agent. It is to be understood that the carrier compound functions to “mask” or “cloak” the heparin-binding agent except in the presence of heparin. In some instances, the carrier compound also does not function to facilitate delivery including in vivo targeting of the heparin-binding agent in vivo.

Examples of carrier compounds include chondroitin sulfate such as chondroitin-4-sulfate and chondroitin-6-sulfate, dermatan sulfate, keratan sulfate, and heparan sulfate. Carrier compounds such as chondroitin sulfate range in molecular weight from about 8 to about 45 kDa, and typically are provided as heterogenous mixtures. The invention contemplates the use of such heterogenous mixtures or mixtures having a smaller molecular weight range, including purified preparations.

The invention contemplates a variety of complexes and particular combinations of heparin-binding agents and carrier compounds. One of ordinary skill in the art will be able to determine one or more optimal carrier compounds for any given heparin-binding agent using routine competition binding assays.

Heparin and Heparin Therapy

The invention provides devices and methods for detecting the presence of heparin and in some cases neutralizing its effects including its anticoagulant effect. Heparin manifests its anticoagulant effect by potentiating the activity of antithrombin (AT), a potent inhibitor of the reactions of the coagulation cascade. AT binds heparin through a high-affinity pentasaccharide sequence present in about one third of heparin molecules. On its own AT is a relatively inefficient inhibitor, but when bound to heparin AT activity is increased up to 1.000-fold. The heparin-AT complex acts to inactivate a number of coagulation enzymes, including factors Ha (thrombin), Xa, IXa, XIa, and XIIa. Of these, thrombin and factor Xa are most responsive to inhibition.

Heparin is a highly sulfated, polyanionic glycosaminoglycan (GAG). Each heparin polymer may be comprised of one or more disaccharide units such as but not limited to 2-0-sulfated iduronic acid and 6-O-sulfated, N-sulfated glucosamine, IdoA(2S)-G1cNS(6S). Pharmaceutical grade heparin is typically derived from animal mucosal tissues such as porcine intestinal tissue and bovine lung tissue. Heparin is typically provided and used as a mixture of polymers ranging in size and thus molecular weight. Naturally occurring (or native) heparin ranges in size from about 3 kDa to about 30 kDa. Pharmaceutical grade heparin typically has a narrower molecular weight range. Low molecular weight heparin has a molecular weight typically less than 10 kDa, or less than 9 kDa, or less than 8 kDa. Examples of low molecular weight heparins are described in published application US 20070287683. As used herein, the term heparin includes without limitation unfractionated heparin, low molecular weight heparin (LMWH) such as enoxaparin (marketed as Lovenox™, Clexane™, Indenox™ or Xaparin™), dalteparin, tinzaparin, bemiparin, certoparin, nadroparin, parnaparin, reviparin, and ardeparin.

In some embodiments, heparin derivatives such as heparinoids are also detected and in some instances neutralized using the methods of the invention.

In still other embodiments, other anticoagulants such as fondaparinux (Arixtra™) and idraparinux can also be detected and/or neutralized using the methods of the invention. Fondaparinux is 2-deoxy-6-O-sulfo-2-(sulfoamino)-α-D-glucopyranosyl-(1→4)—O—β-D-glucopyranuronosyl-(1→4)—O-2-deoxy-3,6-di-O-sulfo-2-(sulfoamino)-α-D-glucopyranosyl-(1→4)—O-2-O-sulfo-α-L-idopyranouronosyl-(1→4)—O-methyl-2-deoxy-6-O-sulfo-2-(sulfoamino)-α-D-glucopyranoside, decasodium salt. Idraparinux is nonasodium (2S,3S,4S,5R,6R)-6-[2R,3R,4S,5R,6R)-6-[(2R,3S,4S,5R,6R)-2-carboxylato-4,5-dimethoxy-6-[(2R,3R,4S,5R,6S)-6-methoxy-4,5-disulfonatooxy-2(sulfonatooxymethyl)oxan-3-yl]oxyoxan-3-yl]oxy-4,5-disulfonatooxy-2-(sulfonatooxymethyl)oxan-3-yl]oxy-4,5-dimethoxy-3-[(2R,3R,4S,5R,6R)-3,4,5-trimethoxy-6-(sulfonatooxymethyl)oxan-2-yl]oxyoxane-2-carboxylate.

Heparin therapy is used to prevent blood clot formation or to prevent extension of existing blood clots. It may be used in a variety of subjects including but not limited to subjects having or at risk of having an acute coronary syndrome (e.g., NSTEMI), atrial fibrillation, deep-vein thrombosis, angina, and/or pulmonary embolism, subjects scheduled to undergo, undergoing, or that have undergone particular surgeries such as cardiovascular surgery including without limitation cardiopulmonary bypass surgery, abdominal surgery, and orthopedic surgery, subjects undergoing extracorporeal life support such as haemofiltration or haemodialysis, and subjects who are schedule to undergo, are undergoing, or that have undergone other medical interventions such as organ transplant.

Subjects

The subjects of the invention include any subject that would be monitored for coagulation activity and/or that would receive anticoagulant therapy. Preferred subjects are humans. Other subjects include companion animals such as dogs and cats, prized livestock and racing thoroughbred horses.

Patient samples as used herein are samples removed from any of subject as described to herein and not intended as limited to human samples. Patient samples include blood samples and plasma samples, both of which may be used in the methods of the invention. The in vitro analysis methods of the invention are performed on “isolated” patient samples including isolated blood samples and isolated plasma samples. Isolated blood samples or isolated plasma samples mean that the sample has been removed from the subject.

The samples to be tested according to the invention include those that are known to be contaminated with heparin or those that may be contaminated with heparin. In some instances, it is not known prior to performing an in vitro coagulation assay whether the sample contains any heparin.

In Vitro Coagulation Assays

The invention contemplates the use of the complexes described herein in a variety of laboratory tests including various coagulation assays. Examples of coagulation assays include without limitation an activated partial thromboplastin time (aPTT) assays, coagulation pathway factor activity assays such as FII, FV, FVIII, FIX, FX, FXI, and FXII activity assays, activated protein C (APC) resistance assays, Bethesda assays, prothrombin time (PT) assays, dilute Russell's viper venom time (dRVVT) assay, hexagonal (II) phase phospholipid-based assay, activated clotting time (ACT) assay, thromboelastograph (TEG) tracing assay, thrombin generation assay, protein C activity assay, protein S activity assay. Each of these assays is known in the art and kits and/or reagents for performing the assays can be obtained from commercial sources such as Precision BioLogic, Stago, Beckman-Coulter, Siemens, Instrumentation Laboratory, Tcoag, and others.

In any of these assays, typically a blood sample will be collected locally or remotely from the testing laboratory. The blood sample is collected in vacu-tubes which may contain oxalate or citrate in order to bind calcium and thereby prevent further coagulation in the sample prior to analysis. The blood sample may be used as is or may be fractionated into a plasma sample.

In the aPTT (also referred to as “partial thromboplastin time” or “PTT”) assay, the sample is then mixed with a phospholipid, an activator such as silica, celite, kaolin or ellagic acid, and calcium. The contact activator is used to stimulate production of Factor XIIa. The phospholipids form complexes that activate Factor X and prothrombin. Calcium is used to reverse the anticoagulant effects of the oxalate or citrate. The mixture is then allowed to form a thrombus (clot) and the time to do so is measured. An abnormal or prolonged aPTT time will vary depending on the population and local normal ranges should be known. However, as a general guideline, values over 39 seconds may be considered abnormal and prolonged according to the invention, and such samples may be candidates for further analysis using the methods of the invention.

In the dilute Russell's viper venom time (dRVVT) assay, the ability of the venom of the Russell's viper to induce thrombosis is measured. Coagulant present in the venom directly activates Factor X, which converts prothrombin into thrombin in the presence of Factor V and phospholipid. The assay conditions typically include low, rate-limiting concentrations of both venom and phospholipid and these typically yield a normal clotting time of about 23-27 seconds. Clotting times that are 30 seconds or longer would therefore be considered prolonged and such samples may be candidates for further analysis using the methods of the invention.

In the activated protein C (APC) resistance (APCR) assay, the plasma sample is first diluted in FV-deficient plasma. An APTT test is performed on the diluted plasma, with and without addition of purified APC. APC degrades the FV in the patient sample and therefore prolongs the APTT. The reported result is the ratio of the clot time in the presence of APC to the clot time in the absence of APC. The addition of APC typically more than doubles the clot time, and therefore a normal APCR ratio is greater than 2.0.

In the prothrombin (PT) test, tissue thromboplastin and calcium are added to a sample such as a plasma sample and the time for clot formation is measured. The assay may be performed manually (e.g., by the tilt tube method), mechanically (e.g., using a fibrometer or a photo-optical instrument). The normal range of time for clot formation using this assay is 10-14 seconds, with the variation due to differences in the thromboplastin used (e.g., the species and tissue source of the thromboplastin). In some instances, in view of the thromboplastin-based variation that may exist, a normalized ratio may be used as the readout instead of an absolute time readout. An example of such a ratio is the international normalized ratio (INR) which is the ratio of the sample prothrombin clotting time to the mean of the normal population prothrombin clotting time, to the power of the ISI, wherein the ISI (International Sensitivity Index) is a measure of the responsiveness of a particular thromboplastin reagent to plasma from patients receiving warfarin. Lower ISI values indicate a more responsive thromboplastin reagent.

In aPTT-based factor activity assays, serial dilutions of a standard reference plasma is mixed with an equal volume of substrate plasma (ie., plasma deficient in the clotting factor that is being assayed) and an aPTT is performed. The test plasma is treated the same way as the reference plasma with serial dilutions mixed with equal volumes of substrate plasma. The clotting times for the aPTT of the plasma mixes are plotted against dilution on Log-Lin graph paper. The relative amount of the factor that is being assayed in the test plasma compared with standard reference material is extrapolated from the graphs. Reference ranges may be expressed as either a percentage or in IU/dl. Many factors, including factors VIII, IX, X, have reference ranges of 50-150%.

In the Bethesda assay, inhibitor to Factor VIII is screened for by mixing serial dilutions of the test plasma with plasma containing a known amount of Factor VIII. A control consisting of normal plasma mixed with buffer (or in the case of the Nijmegen modification, immunodepleted Factor VIII deficient plasma) is taken to represent the 100% value. After 2 hour incubation at 37° C., the residual Factor VIII activity is measured in an aPTT-based Factor VIII assay. The inhibitor concentration is determined by comparing the difference in Factor VIII activity of the test mixture and the control mixture. If the residual factor VIII activity is between 80-100% the sample does not contain an inhibitor.

In the activated clotting time (ACT) assay, fresh whole blood is mixed with an activator such as celite, glass or kaolin and the time for the mixture to form a clot is measured. A normal clotting time usually falls within 70-180 seconds, but varies considerably depending on the method used for the test.

In the protein C (activity) assay test plasma is incubated at 37° C. with phospholipid, a contact activator such as silica, celite, kaolin or ellagic acid, and a protein C activator such as Protac®. After incubation calcium is added to initiate clotting, and the time taken for the mixture to form a clot is measured. From this the protein C level is determined by comparison to a reference curve (normal reference range is 70-140%).

In the protein S (activity) assay test plasma is incubated at 37° C. with protein S deficient plasma, phospholipid, a contact activator such as silica, celite, kaolin or ellagic acid, and either an excess of activated protein C or an activator of protein C such as Protac®. After incubation calcium is added to initiate clotting, and the time taken for the mixture to form a clot is measured. From this the protein S level is determined from a reference curve (normal reference range is 60-140%).

In the hexagonal (II) phase phospholipid-based assay for lupus anticoagulant (LA), test plasma is incubated with an equal volume of buffer in one tube at 37° C. In a second tube, a similar volume of test plasma is incubated with hexagonal (II) phase phospholipid, which inhibits LAs. After incubation, normal plasma is added to both tubes (to correct any possible factor deficiencies) and an aPTT is performed on both. The clotting times for the aPTTs are compared, and if the tube 2 aPTT is shorter than tube 1 by 8 seconds or more, LA is confirmed.

In thromboelastography (TEG), a whole blood sample is placed into a cuvette at 37° C. A sensor shaft connected to a detector system is inserted into the sample. Contact with the walls of the cup or addition of an activator (e.g. celite) to the cup, initiates clot formation between the cup and the sensor. This is detected and a trace generated that provides information on the speed and strength of clot formation.

As will be understood, readouts from these various assays may be an absolute number (e.g., a clotting time) or they may be a ratio of the measured response from the sample being tested to a response from a standard or normal population. In some instances, it will be important to know the normal readout or normal range. Normal coagulation activity or coagulation level will typically depend upon the readout of each particular assay. The normal coagulation activity or coagulation level may also depend on the subject, as there may be differences for example between male and female subjects and/or between subjects of different ages. The normal coagulation activity or coagulation level may also vary depending on the health of the subject, and any treatments the subject is receiving. Those of ordinary skill in the art of coagulation assays will be familiar with normal coagulation activity or coagulation levels from a variety of assays including those specifically recited herein.

Abnormal coagulation activity or coagulation levels, in the context of this invention, refer to coagulation activity or coagulation level that is below normal (i.e., an abnormal reading indicates that the sample is less able to coagulate and therefore typically takes longer to coagulate). As will be understood by those of ordinary skill in the art, absolute values of abnormal coagulation activity will depend upon absolute values of normal activity.

As an example, various assays described herein have as their readout clotting time, including the aPTT assay. In that assay, a normal aPTT clotting time is generally about 35 seconds with a range of about 25-39 seconds.

An abnormal (or prolonged) clotting time in some instances therefore may be a clotting time in excess of the upper end of the normal range of an assay. In some instances, an abnormal clotting time may be a clotting time that is at least 1 second, at least 2 seconds, at least 3 seconds, at least 4 seconds, at least 5 seconds, at least 6 seconds, at least 7 seconds, at least 8 seconds, at least 9 seconds, at least 10 seconds, at least 20 seconds, at least 40 seconds, at least 50 seconds, at least 60 seconds, at least 70 seconds, at least 80 seconds, at least 90 seconds, at least 100 seconds, at least 110 seconds, at least 120 seconds, at least 130 seconds, at least 140 seconds, at least 150 seconds, at least 160 seconds, at least 170 seconds, at least 180 seconds, at least 190 seconds, at least 200 seconds, at least 210 seconds, at least 220 seconds, at least 230 seconds, at least 240 seconds, or more, longer than the upper end of the normal clotting time range. In some instances, an abnormal clotting time may be a clotting time that is 2%, 3%, 4%, 5% or more, longer than the upper end of the normal clotting time range.

In some instances, a typical abnormal clotting time, as an absolute value, ranges from about 40 seconds to greater than 240 seconds (depending on the assay), including about or at least 20 seconds, about or at least 40 seconds, about or at least 50 seconds, about or at least 60 seconds, about or at least 70 seconds, about or at least 80 seconds, about or at least 90 seconds, about or at least 100 seconds, about or at least 110 seconds, about or at least 120 seconds, about or at least 130 seconds, about or at least 140 seconds, about or at least 150 seconds, about or at least 160 seconds, about or at least 170 seconds, about or at least 180 seconds, about or at least 190 seconds, about or at least 200 seconds, about or at least 210 seconds, about or at least 220 seconds, about or at least 230 seconds, or about or at least 240 seconds, or more.

Accordingly, samples characterized by abnormal clotting times, as described herein or as otherwise identified in the art, may be candidates for use with the compositions of the invention. The foregoing is intended for exemplary purposes and one of ordinary skill in the art will appreciate that its teaching is to be extrapolated to any of the assays used to assess coagulation activity.

It is to be understood that the invention contemplates, in some instances, performing any of the foregoing assays in the absence of a complex and then performing the same assay again with another aliquot from the same patient sample but in the presence of the complex. A difference in the readouts between the two assays is indicative of the presence of heparin. Some aspects of the invention therefore allow an end user to detect the presence of heparin even without performing the assay using a “normal” sample or without knowing the normal readout. The invention contemplates that the differences between assays performed in the presence or absence of the complex may be reported as absolute numbers or as ratios. If the ratio represents the ratio of clotting time in the absence of the complex to clotting time in the presence of the complex, then a ratio of about 1 will indicate absence of heparin in the sample while a ratio greater than 1 will indicate presence of heparin in the sample. Depending the assay, ratios of 1.5, 2, 3, 4, 5, 6, 7, 8, 9, 10 or greater may indicate the presence of heparin.

According to these and other methods provided herein, prolonged clotting times may be ascribed to the presence of heparin (i.e., the use of the compositions of the invention allow accurate analytical coagulation readouts to be obtained by reducing or eliminating heparin-associated anti-coagulation activity). The ability to reduce clotting time to within a normal range using the compositions of the invention can also be useful in excluding certain conditions as the cause of the prolonged clotting time. Such conditions include without limitation Von Willebrand disease, end-stage liver failure, coagulation factor deficiency leading to hemophilia, or the presence of antiphospholipid antibody such as lupus anticoagulant. Alternatively, if clotting time is not reduced to a normal range through the use of the compositions of the invention, other causative factors may be implicated including without limitation one of the foregoing conditions.

Detectable Labels

In some embodiments, the invention contemplates detectably labeling the heparin-binding agent and/or the carrier compound. Such labeling is useful in methods of the invention that measure binding of heparin (or heparin derivatives) to a heparin binding agent and/or dissociation of the carrier compound from the heparin-binding agent in the presence of heparin or a heparin derivative. The heparin-binding agent may be detectably labeled by linking it to a detectable label. Similarly, the carrier compound may be detectably labeled by linking it to a detectable label.

Detectable labels, as used herein, are agents that can be detected directly or indirectly. They may be light emitting, energy accepting, fluorescent, radioactive, or quenching. The detectable labels may be but are not limited to chromophores such as but not limited to phycobilins, and pyrenes; chemiluminescent compounds such as but not limited to aminophthalhydrazides, acridinium esters, and peroxyoxalates; fluorophores such as but not limited to fluorescein (e.g., fluorescein succinimidyl ester), TRITC, rhodamine, tetramethylrhodamine, R-phycoerythrin, Cy-3, Cy-5, Cy-7, Texas Red, Phar-Red, allophycocyanin (APC); radioactive isotopes such as but not limited to P³² or H³; epitope tags such as but not limited to the FLAG or HA epitope; enzyme tags such as but not limited to alkaline phosphatase, horseradish peroxidase, β-galactosidase, etc.

In some embodiments, a signal is detectable when the heparin-binding agent is bound to the carrier compound and this signal is reduced or eliminated when the carrier compound dissociates from the heparin-binding agent. Accordingly, a decreased signal in the presence of a patient sample (or another sample being tested) indicates presence of heparin or a heparin derivative in the sample. As an example, this may be achieved if the heparin-binding agent and carrier compound are bound to members of a fluorescence transfer pair, such as members of a FRET pair. The heparin-binding agent may be linked to the fluorescence donor and the carrier compound may be linked to the fluorescence acceptor (or vice versa). In either arrangement, a stronger fluorescent signal is observed when the heparin-binding agent and the carrier compound are complexed to each other because the fluorescence transfer can only occur when the donor and acceptor are proximal to each other. A reduced (including no) fluorescent signal is observed when the heparin-binding compound dissociates from the carrier compound.

In other embodiments, signal is detected when the heparin-binding agent is bound to heparin or a heparin derivative but not when it is bound to the carrier compound. In these embodiments, a higher signal in the presence of a patient sample (or another sample being tested) indicates the presence of heparin or a heparin derivative. As an example of such an arrangement, the heparin-binding agent may be linked to a fluorophore and the carrier compound may be linked to a quencher. When the heparin-binding agent and the carrier compound are complexed to each other, a reduced (or no) signal is detectable from the fluorophore. When the heparin-binding agent and the carrier compound are not complexed to each other (e.g., in the presence of heparin or a heparin derivative), the signal is detectable.

Pharmaceutical Compositions, Formulations, Effective Amounts

The invention further provides a pharmaceutical composition or preparations comprising a complex of a heparin-binding agent and a carrier compound.

The pharmaceutical preparations are administered in effective amounts. For therapeutic applications, it is generally that amount sufficient to achieve a medically desirable result, including for example the amount sufficient to reduce plasma heparin levels in a subject. The effective amount may depend upon the mode of administration, and the desired outcome. It may also depend upon the stage and/or severity of the condition, the age and physical condition of the subject being treated, the presence and/or nature of concurrent therapy, the duration of the treatment, and like factors within the knowledge and expertise of the medical practitioner. For prophylactic applications, it is that amount sufficient to delay the onset of, inhibit the progression of, or halt altogether the particular condition being prevented, and may be measured by the amount required to prevent the onset of symptoms.

The complexes of the invention can be administered to a subject in need thereof in combination with concurrent therapy for neutralizing heparin. The concurrent therapy may be invasive or non-invasive (e.g., drug therapy). Examples of drug therapies for neutralizing heparin include but are not limited to protamine (e.g., protamine sulfate). It is contemplated that in some instances the drug therapies may be administered in amounts which are not capable of reducing heparin levels when used alone but which have an effect on heparin when used in combination with the complexes of the invention. The complex may be formulated with such secondary therapeutic agents or they may be formulated separately. They may be administered at the same time or at separate times. For example, the complex of the invention may be administered before, and/or with, and/or after the secondary therapeutic agent. Alternatively, the secondary therapeutic agent may be administered before, and/or with, and/or after the complex of the invention.

The complexes of the invention may be administered alone or in combination with the above-described drug therapies as part of a pharmaceutical composition. Such a pharmaceutical composition may include the complexes of the invention in combination with any standard physiologically and/or pharmaceutically acceptable carriers which are known in the art. The compositions should be sterile and contain a therapeutically effective amount of the complex in a unit of weight or volume suitable for administration to a patient.

The term “pharmaceutically-acceptable carrier” as used herein means one or more compatible solid or liquid filler, diluents or encapsulating substances which are suitable for administration into a human or other animal. The term “pharmaceutically acceptable” means a non-toxic material that does not interfere with the effectiveness of the biological activity of the active ingredients. Pharmaceutically acceptable further means a non-toxic material that is compatible with a biological system such as a cell, cell culture, tissue, or organism. The term “carrier” denotes an organic or inorganic ingredient, natural or synthetic, with which the active ingredient is combined to facilitate the application. The characteristics of the carrier will depend on the route of administration. The components of the pharmaceutical compositions also are capable of being commingled with the agents of the present invention, and with each other, in a manner such that there is no interaction which would substantially impair the desired pharmaceutical efficacy. The pharmaceutically acceptable carrier must be sterile for in vivo administration. Physiologically and pharmaceutically acceptable carriers include diluents, fillers, salts, buffers, stabilizers, solubilizers, and other materials which are well known in the art.

Compositions suitable for parenteral administration conveniently comprise a sterile aqueous preparation of the complexes, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulations suitable for oral, subcutaneous, intravenous, intramuscular, etc. administrations can be found in Remington's Pharmaceutical Sciences, Mack Publishing Co., Easton, Pa.

The in vivo methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, inhalation, or parenteral routes. The term “parenteral” includes subcutaneous, intravenous, intramuscular, or intraperitoneal administration.

The pharmaceutical compositions may conveniently be presented in unit dosage form and may be prepared by any of the methods well-known in the art of pharmacy. All methods include the step of bringing the complexes into association with a carrier which constitutes one or more accessory ingredients. Compositions include suspensions in aqueous liquids or non-aqueous liquids such as a syrup, elixir or an emulsion.

Kits

The invention contemplates kits that comprise one or more reagents for performing a coagulation assay, such as any of the coagulation assays described herein, and a container comprising the complex of the invention.

The invention also contemplates kits that comprise evacuated vacuum venipuncture collection tubes that comprise the complex of the invention, for example, in solution, solid, or semi-solid form, and/or as a coating to an interior surface of the tube.

The invention also contemplates kits that comprise the complex bound to a solid support. Examples of solid supports include dipsticks, filters such as nitrocellulose filters, microspheres (e.g., glass, silica or polystyrene microspheres). The solid support is preferably not an affinity chromatography column.

### EXAMPLES

The following Examples demonstrate the efficacy of an exemplary complex comprising PF4 and chondroitin-4-sulfate.

A reagent for neutralizing patient plasma samples suspected of heparin contamination (i.e. have unexplained prolonged aPTTs) is provided. The neutralizing reagent fully reverses any heparin-associated anticoagulant activity, but otherwise does not interfere with the clotting time thereby allowing for an accurate aPTT to be recorded. The active ingredient of the neutralizing reagent is a naturally occurring protein called platelet factor 4 (PF4). PF4 is a moderately cationic (pI=8.7) polypeptide comprised of 70 amino acids with a molecular weight of 7.8 kDa. Synthesized in megakaryocytes, PF4 is ultimately stored in the α-granules of blood platelets for later secretion through platelet activation and aggregation. Under physiological conditions, PF4 exists as a tetramer of identical subunits bound to a proteoglycan carrier consisting of four chondroitin-4-sulfate chains covalently linked to a single polypeptide that is also secreted by the platelets (Huang et al. 1982). While the exact physiological role of PF4 is not yet fully understood, it has been implicated in diverse biological processes including the inhibition of endothelial cell growth and angiogenesis, chemotactic attraction of neutrophils and monocytes, megakaryocyte growth and maturation, and immune system regulation (Maione et al. 1991; see U.S. Pat. Nos. 5,112,946, 5,317,011, 5,436,222, 5,304,542, 5,284,827, and 4,702,908).

The PF4-based reagent may be used for the in vitro neutralization of heparin in samples as assessed by the aPTT assay. To ensure an accurate aPTT result the reagent must act specifically to reverse only anticoagulant activity associated with heparin, and otherwise have no effect on the plasma clotting time. It was found however that increasing amounts of PF4 exert a paradoxical anti-coagulant effect on non-heparinized plasma resulting in a gradual prolongation of the aPTT (FIG. 2). This phenomenon might be due to PF4-induced stimulation of the thrombin-mediated cleavage of protein C to generate the potent anticoagulant activated protein C (APC). APC acts to inactivate coagulation Factors Va and VIIIa by proteolysis. Stimulation of APC generation by PF4 is reversed by heparin or chondroitin sulfate. Alternatively, PF4 delays initiation of the intrinsic pathway of coagulation by inhibiting the contact activation of Factor XII and prekallikrein. Activation is promoted through the binding of the coagulation factors to negatively charged surfaces, and neutralization of the negative charges by PF4 could interfere with this process. This effect is inhibited by heparin or by anti-PF4 antiserum.

It was hypothesized that a heparin-like carrier compound incorporated in the neutralizer reagent would bind and sequester free PF4 and prevent it from interfering with the clotting time of the plasma sample. According to the invention, it was found that PF4-associated anti-coagulation does not occur when PF4 is bound to heparin (i.e., the aPTT is fully corrected to baseline during heparin neutralization, as shown in FIG. 1). For PF4 to be available for heparin neutralization however it must readily dissociate from the carrier compound and transfer to heparin when present in the plasma sample. This should occur if the carrier compound has a lower binding affinity relative to heparin for the PF4.

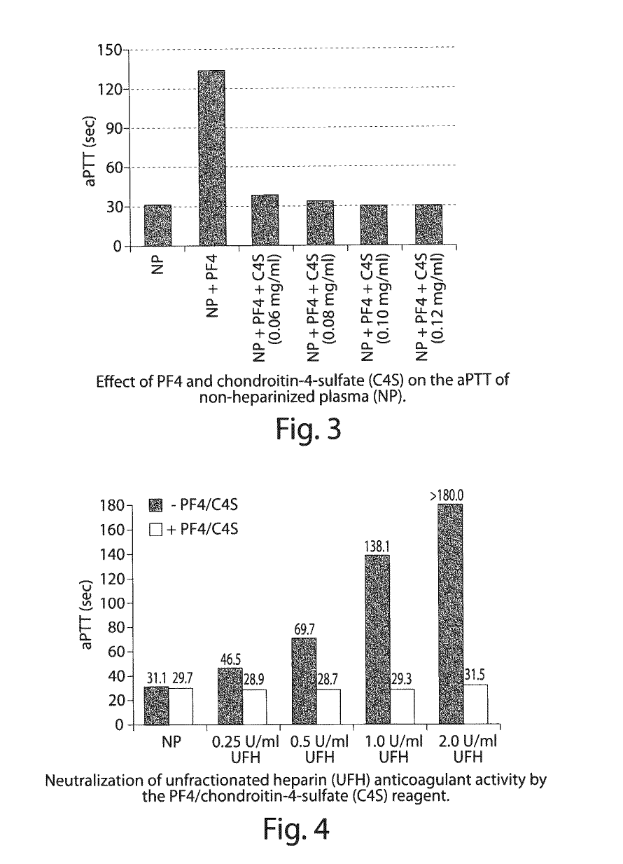

PF4 binds various heparin-like, sulfated GAGs with a binding affinity that correlates with the degree of sulfation of the compound (i.e., heparin>heparan sulfate>dermatan sulfate>chondroitin-6-sulfate>chondroitin-4-sulfate). Chondroitin-4-sulfate was used due to its low relative affinity for PF4, and while it does have anti-coagulant activity itself, its potency is much lower than that of heparin. As shown, pre-binding of PF4 to chondroitin-4-sulfate fully masks the proteins anticoagulant activity in non-heparinized plasma, and restores the aPTT to the baseline level (FIG. 3). Importantly, PF4/chondroitin-4-sulfate complex is still capable of fully reversing the heparin anticoagulant activity of plasma containing up to 2 U/mL of UFH (FIG. 4).

### REFERENCES

Bernabei et al. (1995) Reversal of heparin anticoagulation by recombinant platelet factor 4 and protamine sulfate in baboons during cardiopulmonary bypass. J. Thorac. Cardiovasc. Surg. 109:765-771.
Bjornsson et al. (1982) The anticoagulant effect of chondroitin-4-sulfate. Thromb. Res. 27:15-21.
Cook et al. (1992) Platelet factor 4 efficiently reverses heparin anticoagulation in the rat without adverse effects of heparin-protamine complexes. Circulation 85:1102-1109.
Cumming et al. (1986) In vitro neutralization of heparin in plasma prior to the activated partial thromboplastin time test: an assessment of four heparin antagonists and two anion exchange resins. Thromb. Res. 41:43-56.
Dehmer et al. (1995) Reversal of heparin anticoagulation by recombinant platelet factor 4 in humans. Circulation 91:2188-2194.
Dumenco et al. (1988) Inhibition of the activation of Hageman factor (factor XII) by platelet factor 4. J. Lab. Clin. Med. 112:394-400.
Handin and Cohen (1976) Purification and binding properties of human platelet factor four. J. Biol. Chem. 251:4273-4282.
Huang et al. (1982) Proteoglycan carrier of human platelet factor 4. J. Biol. Chem. 257:11546-11550.
Korutla et al. (1994) Evaluation of recombinant platelet factor 4 and protamine sulfate for heparin neutralization: clotting and clearance studies in rat. Thromb. Haemost. 71:609-614.
Maione et al. (1990) Inhibition of angiogenesis by recombinant human platelet factor-4 and related peptides. Science 247:77-79.
Preston et al. (2009) Platelet factor 4 impairs the anticoagulant activity of activated protein C. J. Biol. Chem. 284:5869-5875.
Scully et al. (1980) Inhibition of contact activation by platelet factor 4. Thromb. Res. 20:461-466.
Slungaard and Key (1994) Platelet factor 4 stimulates thrombomodulin protein C-activating cofactor activity. J. Biol. Chem. 269:25549-25556.
Slungaard et al. (2003) Platelet factor 4 enhances generation of activated protein C in vitro and in vivo. Blood 102:146-151.
Weerasinghe et al. (1983) A platelet derived inhibitor of plasma prekallikrein activation. Thromb. Res. 32:519-529.
Weerasinghe et al. (1984) Inhibition of the cerebroside sulphate (sulphatide)-induced contact activation reactions by platelet factor four. Thromb. Res. 33:625-631.
Yang and Rezaie (2007) Calcium-binding sites of the thrombin-thrombomodulin-protein C complex: Possible implications for the effect of platelet factor 4 on the activation of vitamin K-dependent coagulation factors. Thromb. Haemost. 97:899-906.
Zucker and Katz (1991) Platelet factor 4: production, structure, and physiologic and immunologic action. Proc. Soc. Exp. Biol. Med. 198:693-702.
U.S. Pat. No. 4,195,072: Stabilized platelet factor 4 immunoassay standards
U.S. Pat. No. 4,702,908: Composition containing platelet factor 4 and method for restoring suppressed immune responses
U.S. Pat. No. 5,112,946: Modified PF4 compositions and methods of use
U.S. Pat. No. 5,204,321: Heparin neutralization with platelet factor 4
U.S. Pat. No. 5,284,827: Systemic treatment of metastatic cancer with platelet factor 4
U.S. Pat. No. 5,304,542: Use of platelet factor 4 to inhibit osteoblast proliferation
U.S. Pat. No. 5,436,222: Use of platelet factor 4 to treat inflammatory diseases
U.S. Pat. No. 5,464,815: Inhibition of heparin binding
U.S. Pat. No. 5,482,923: Heparin neutralization with platelet factor 4 fragments
U.S. Pat. No. 5,317,011: Cloning and expression of a variant gene of platelet factor 4 and
compositions thereof to modulate immune response
U.S. Pat. No. 5,585,095: Method to enhance thrombomodulin APC generation using cationic proteins

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method comprising
adding, to a patient sample in vitro, a composition comprising a complex of a heparin-binding agent and a carrier compound, wherein the heparin-binding agent has a higher binding affinity for heparin than for the carrier compound, and
measuring coagulation activity in the patient sample.

2. The method of claim 1, wherein coagulation activity in the presence of the complex is compared to coagulation activity in the absence of the complex.

3. The method of claim 2, wherein coagulation activity that is greater in the presence of the complex than in the absence of the complex indicates presence of heparin or a heparin derivative in the patient sample.

4. A method comprising
performing a coagulation assay, on a patient sample, in the presence of a heparin-binding agent complexed to a carrier compound, wherein the heparin-binding agent complexed to the carrier compound is allogeneic to the patient sample and wherein the heparin-binding agent has a higher binding affinity for heparin than for the carrier compound.

5. The method of claim 4, further comprising performing another coagulation assay, using another aliquot of the patient sample, in the absence of the heparin-binding agent complexed to the carrier compound.

6. The method of claim 4 or 5, further comprising comparing results from the coagulation assay in the presence of the heparin-binding agent complexed to the carrier compound to results from a coagulation assay performed in the absence of the heparin-binding agent complexed to the carrier compound.

7-8. (canceled)

9. The method of claim 1 or 4, wherein the patient sample is characterized as having a prolonged clotting time, in the absence of the heparin-binding agent complexed to the carrier compound.

10-12. (canceled)

13. The method of claim 1 or 4, wherein the heparin-binding agent is a heparin-binding protein.

14-20. (canceled)

21. The method of claim 1 or 4, wherein the heparin-binding agent is platelet factor 4 (PF4).

22. The method of claim 1 or 4, wherein the heparin-binding agent is a heparin-binding platelet factor 4 (PF4) fragment.

23. The method of claim 1 or 4, wherein the heparin-binding agent is poly-L-lysine, protamine sulfate or hexadimethrine bromide (Polybrene®).

24-26. (canceled)

27. The method of claim 1 or 4, wherein the carrier compound is a glycosaminoglycan.

28. (canceled)

29. The method of claim 1 or 4, wherein the carrier compound is chondroitin sulfate.

30-33. (canceled)

34. The method of claim 1 or 4, wherein the heparin-binding agent is platelet factor 4 (PF4) and the carrier compound is chondroitin sulfate.

35. (canceled)

36. The method of claim 1 or 4, wherein the coagulation assay is an activated partial thromboplastin time (aPTT) assay, an FII, FV, FVIII, FIX, FX, FXI or FXII activity assay, a Bethesda assay, a dilute Russell's viper venom time (dRVVT) assay, an activated clotting time (ACT) assay, a thromboelastograph (TEG) tracing, or a thrombin generation assay.

37-47. (canceled)

48. The method of claim 1 or 4, wherein the patient sample is a blood sample or a plasma sample.

49-57. (canceled)

58. The method of claim 1 or 4, wherein heparin is unfractionated heparin, low molecular weight heparin, or a heparinoid.

59-80. (canceled)

81. A composition comprising
a synthetic complex of a heparin-binding agent and a carrier compound wherein the heparin-binding agent has a lower binding affinity for the carrier compound than heparin, and a preservative.

82-84. (canceled)

85. A composition comprising
a heparin-binding agent,
a carrier compound wherein the heparin-binding agent has a lower binding affinity for the carrier compound than heparin, and
a patient sample allogeneic to the heparin-binding agent.

86. The composition of claim 81 or 85, wherein the heparin-binding agent is a heparin-binding protein, or a heparin-binding polymer.

87-93. (canceled)

94. The composition of claim 81 or 85, wherein the heparin-binding agent is platelet factor 4 (PF4).

95. The composition of claim 81 or 85, wherein the heparin-binding agent is a heparin-binding fragment of platelet factor 4 (PF4).

96. The composition of claim 81 or 85, wherein the heparin-binding agent is poly-L-lysine, protamine sulfate, or hexadimethrine bromide (Polybrene®).

97-99. (canceled)

100. The composition of claim 81 or 85, wherein the carrier compound is a glycosaminoglycan.

101. (canceled)

102. The composition of claim 81 or 85, wherein the carrier compound is chondroitin sulfate.

103. The composition of claim 81 or 85, wherein the heparin-binding agent is platelet factor 4 (PF4) and the carrier compound is chondroitin sulfate.
